(19) United States
(12) Patent Application Publication
(10) Pub. No.: US 2004/0033536 A1
(43) Pub. Date: Feb. 19, 2004

(54) Methods for identifying and using insecticides

(75) Inventors: Karen A. Mesce, St. Paul, MN (US); Kathleen A. Klukas, St. Paul, MN (US)

Correspondence Address: MUETING, RAASCH & GEBHARDT, P.A., P.O. BOX 581415, MINNEAPOLIS, MN, 55458, US

(73) Assignee: Regents of the University of Minnesota, Minneapolis, MN

(21) Appl. No.: 10/422,347

(22) Filed: Apr. 24, 2003

Related U.S. Application Data
(60) Provisional application No. 60/375,244, filed on Apr. 24, 2002.

(51) Int. Cl. G01N 33/567; A61K 49/00

(52) U.S. Cl. 435/7.2; 424/9.2

(57) ABSTRACT

The present invention provides methods for identifying compounds that prevents ecdysis. Also provided by the present invention are methods for preventing ecdysis, and for killing insects.

This application claims the benefit of U.S. Provisional Application Serial No. 60/375,244, filed Apr. 24, 2002, which is incorporated by reference herein.

### BACKGROUND

Insect pests are a major factor in the loss of food crops and forest defoliation. Managing such pests of economic importance, without compromising the environment and the health of humans and beneficial insects, is a primary objective of sustainable agriculture and the forest industry. Presently, the insecticide market is dominated by three classes of compounds (pyrethroids, carbamates, and organophosphates), which act on insect neural tissues (Benson, In: Neurotox'91 Molecular Basis of Drug &Pesticide Action (Duce, I. R., ed), pp 57-70. London and New York: Elsevier Applied Science (1991)). Natural (nicotine) and commercial insecticides (Imidacloprid) have proven how very well a receptor's ligand-binding site can be effectively targeted. Unfortunately, many commercially based insecticides bind to the same sites shared by many vertebrate and invertebrate species (Bloomquist, Annu. Rev. Entomol., 41, 163-90 (1996)). Ecdysis, or the shedding of the old cuticle, is a fundamental process involved in insect growth and development, for example, facilitating the development to a more mature stage of development. It has long been recognized that ecdysis represents a particularly vulnerable stage in the insect's life, and interfering with the precisely orchestrated series of signaling events underlying ecdysis can have dire consequences for the insect's survival.

### SUMMARY OF THE INVENTION

The present invention represents an advance in the art of controlling insects. The present invention provides methods for identifying a compound that prevents ecdysis. The method includes providing a compound that binds a polypeptide. In some aspects, the polypeptide is a mouse IgG. The compound may bind an Fe domain of the mouse IgG antibody, an F(ab′)2 domain of the mouse IgG antibody, or an Fab domain of the mouse IgG antibody. In other aspects, the polypeptide is obtainable from an insect, and the polypeptide includes an amino terminal sequence of X₁X₂A(Q/K)(Q/K)GVD(L/1)SR (SEQ ID NO: 1), where X₁ is either alanine, leucine, proline, or serine and X₂ is either alanine, leucine, proline, or serine, and a molecular weight of between about 25 kDa and about 29 kDa. The method further includes exposing an insect to an effective amount of the compound, and observing ecdysis in the insect. Failure of the insect to complete ecdysis indicates the compound prevents ecdysis. The compound may be a polypeptide. Preferably, the compound is an antibody, for instance a polyclonal antibody or a monoclonal antibody. Preferably, the insect is at a larval stage of development.

The present invention also provides methods for preventing ecdysis. The methods include exposing an insect to an effective amount of a compound that binds a polypeptide, wherein the insect does not complete ecdysis. In some aspects, the polypeptide is a mouse IgG, and in other aspects, the polypeptide is obtainable from an insect, and the polypeptide includes an amino terminal sequence of X₁X₂A(Q/K)(Q/K)GVD(L/1)SR (SEQ ID NO:1), where X₁ is either alanine, leucine, proline, or serine and X₂ is either alanine, leucine, proline, or serine, and a molecular weight of between about 25 kDa and about 29 kDa.

The present invention further provides methods for killing an insect. The methods include exposing an insect to an effective amount of a compound that binds a polypeptide, wherein the compound causes the insect to die after failing to complete ecdysis. In some aspects, the polypeptide is a mouse IgG, and in other aspects, the polypeptide is obtainable from an insect, and the polypeptide includes an amino terminal sequence of X₁X₂A(Q/K)(Q/K)GVD(L/1)SR (SEQ ID NO:1), where X₁ is either alanine, leucine, proline, or serine and X₂ is either alanine, leucine, proline, or serine, and a molecular weight of between about 25 kDa and about 29 kDa.

Unless otherwise specified, “a,” “an,” “the,” and “at least one” are used interchangeably and mean one or more than one.

### DETAILED DESCRIPTION OF PREFERRED EMBODIMENTS OF THE INVENTION

The present invention provides methods for identifying compounds that prevent ecdysis in an insect, preferably, kill the insect. The method includes providing a compound that binds to a polypeptide, exposing an insect to an effective amount of a compound, and observing ecdysis in the insect. The failure of the insect to complete ecdysis indicates the compound prevents ecdysis. As used herein, “bind” refers to the ability of a compound to interact, either directly or indirectly, with a polypeptide. The interaction can be, for instance, an ionic bond, a hydrogen bond, a Van der Waals force, or a combination thereof. Without intending to be limiting, a compound useful in the methods of the present invention can be, for instance, an organic compound, an inorganic compound, a polypeptide, a non-ribosomal polypeptide, or a polyketide. Preferably, a compound is a polypeptide. “Polypeptide” as used herein refers to a polymer of amino acids linked by peptide bonds and does not refer to a specific length of a polymer of amino acids. Thus, for example, the terms peptide, oligopeptide, protein, and enzyme are included within the definition of polypeptide. This term also includes post-expression modifications of a polypeptide, for example, glycosylations, acetylations, phosphorylations and the like. A polypeptide can be obtained directly from a natural source, or can be prepared with the aid of recombinant, enzymatic, or chemical techniques.

In some aspects, the polypeptide to which the compound binds contains one or more three dimensional motifs that are present in an immunoglobulin G (IgG) obtained from a mouse. The motifs common to the mouse IgG can belong to any subclass. Preferably, the polypeptide is a mouse IgG. Identifying a compound that binds to a mouse IgG can be accomplished using methods known to the art. Such methods include, for instance, western immunoblot where the mouse IgG has been resolved on a gel, transferred to a membrane, and the membrane exposed to the compound to determine if the compound binds to the IgG. Alternatively, the compound may be resolved on the gel, transferred to a membrane, and the membrane exposed to a mouse IgG. Other methods include a version of the technique often referred to as affinity chromatography. For instance, a mouse IgG can be attached to a matrix, for instance a matrix present in a column, and the mouse IgG exposed to the compound. A compound useful in the present invention may bind to either an F(ab′)2 fragment of a mouse IgG, an Fab fragment of a mouse IgG, or an Fc fragment of a mouse IgG, or a combination thereof. F(ab′)2 fragments, Fab fragments, and Fc fragments of mouse IgG are commercially available. An example of a compound that binds a mouse IgG is an anti-mouse IgG antibody. Such an anti-mouse IgG antibody may be polyclonal or monoclonal, preferably polyclonal. Further, F(ab′)2 fragments or Fab fragments can be prepared from such an anti-mouse IgG antibody using methods routine in the art, and used in the present methods. Polyclonal and monoclonal antibodies obtained from various species that bind to mouse IgG are commercially available, as are F(ab′)2 fragments or Fab fragments. Typically, an antibody that can bind a mouse IgG antibody is one that that interacts only with the portion (also referred to as an epitope) of the antigen that induced the synthesis of the antibody, or interacts with a structurally related epitope.

In other aspects, the polypeptide to which the compound binds is obtainable from an insect, and is referred to herein as “mIgG-like polypeptide” or “mouse IgG-like polypeptide.” The mIgG-like polypeptide has a molecular weight of between about 25 kilodaltons (kDa) to about 29 kDa, preferably, about 27 kDa, and includes the amino terminal sequence of X₁X₂A(Q/K)(Q/K)GVD(L/1)SR (SEQ ID NO:1), where X₁ is either alanine, leucine, proline, or serine and X₂ is either alanine, leucine, proline, or serine, more preferably, where X₁ is either alanine or leucine and X₂ is either alanine or leucine, or X₁ is either proline or serine and X₂ is either proline or serine, most preferably, X₁ is either alanine or leucine and X₂ is either alanine or leucine. This mIgG-like polypeptide is also bound by antibody that binds mouse IgG. Another characteristic of the mIgG-like polypeptide is that it is bound by Protein G, a commercially available polypeptide that binds to the constant region of IgG molecules from many species, including mouse. The mIgG-like polypeptide is present on a discrete population of neurosecretory neurons in larvae and pupae of most insects, the neurosecretory lateral neurons (also known as the cell 27s in Manduca sexta) and the interneurons (also known as the In-704s in M. sexta) (Klukas et al., Microscopy Res. Tech., 35, 242-264 (1996)).

The IigG-like polypeptide can be obtained by removal of neurons of the central nervous system from an insect, preferably M. sexia. Preferably, the insect is in the larval or pupal stage of development, most preferably, the larval stage. Preferably, the ventral nerve cords of insects are removed. A method for isolating the mIgG-like polypeptide from an insect is described at Example 2. Briefly, the nerve cords are placed on ice in a saline solution, transferred to a solution, for instance, hypomillonigs, and disrupted under conditions to allow separation of membrane-associated proteins from cytoplasmic protein. After separation, for instance by centrifugation, the membrane-associated proteins resuspended in buffer containing between about 7 grams/liter and about 8 grams/liter Trizma, preferably about 7.58 g/liter Trizma, and between about 0.5% and about 1.5% Nodinet P-40, preferably about 1% Nodinet P-40. This suspension is disrupted, preferably by sonication, and after separation of the solubilized and unsolubilized proteins, the soluble fraction is exposed to a polypeptide that binds the mIgG-like polypeptide, for instance Protein G or an anti-mouse IgG, under conditions that allow the mIgG-like polypeptide to bind. Elution of the proteins that have bound to the protein G results in a population of proteins enriched for the mouse IgG-like polypeptide. An “isolated” polypeptide means a polypeptide that has been either removed from its natural environment, produced using recombinant techniques, or chemically or enzymatically synthesized. Preferably, a polypeptide is purified, i.e., essentially free from any other polypeptide and associated cellular products or other impurities.

Identifying a compound that binds to an IigG-like polypeptide can be accomplished using methods known to the art. Such methods include, for instance, western immunoblot where the mIgG-like polypeptide has been resolved on a gel, transferred to a membrane, and the membrane is exposed to the compound to determine if the compound binds to the mIgG-like polypeptide. Alternatively, the compound may be resolved on the gel, transferred to a membrane, and the membrane exposed to an mIgg-like polypeptide. Other methods include a version of the technique often referred to as affinity chromatography. For instance, mIgG-like polypeptide can be attached to a matrix, for instance a matrix present in a column, and the mIgG-like polypeptide exposed to the compound. Preferably, a compound that binds an mIgG-like polypeptide is antibody. Such an antibody may be polyclonal or monoclonal, preferably polyclonal. Further, such an antibody can be F(ab′)2 fragments or Fab fragments. Methods of making polyclonal and monoclonal antibodies are known to the art and are routine. See, for example, Antibodies: A Laboratory Manual, Harlow and Lane, eds., Cold Spring Harbor Laboratory Press: Cold Spring Harbor, N.Y., 1988).

An insect is exposed to an effective amount of a compound that binds a mouse IgG or an mIgG-like polypeptide. Exposure of the insect to the compound includes, for instance, injection, infusion, and oral administration. In those aspects of the present invention directed to identifying compounds that prevent ecdysis, preferably the compound is administered orally, by injection or by infusion, more preferably, by injection or by infusion, most preferably, by injection. As used herein, an “effective amount” is an amount effective to prevent an insect from undergoing a complete ecdysis. When administered by injection, the compound is injected into the hemolymph (blood) of the insect. A commercially available needle or a glass needle can be used. Typically, a commercially available needle is used on insects where the volume to deliver is at least about 20 microliters (μl), and a glass needle is used with insects where the volume to inject is no greater than about 500 μl. Typically, the amount injected into an M. sexta insect at the fifth instar is typically from between about 25 μl to about 250 μl, and the amount injected into an Indian meal moth at the fifth instar is typically between about 1 μl to about 20 μl. The upper range of the volume that can be injected into an insect of a particular developmental stage can be determined empirically by a skilled person. When administered orally, a solution of the compound can be applied to the food source of the insect. When administered by infusion, about the same volume is injected into the insect, but the injection occurs over a time period of at least about 10 hours, at a rate of about 2 μl per hour. Machines that can be used to infuse an insect are commercially available, and an example is the syringe pump model 352 available from Sage Instruments Division of Orion Research Inc. (Freedom, Calif.).

The insects used in the methods for identifying a compound that prevents ecdysis are in the larval or pupal stage of development, more preferably, the larval stage. The insect can be exposed to a compound and then observed for ecdysis. Preferably, the insects used are staged such that the approximate time that the insect will begin to ecdyse can be predicted. Methods for staging a population of insects are routine in the art. Preferably, the compound is administered at between least about 1 hour to several days before the insect is expected to begin to ecdyse, and as the insect observed as it nears the expected time of ecdysis. Observing ecdysis may include noting the beginning of a series of coordinated abdominal movements. These movements result in ecdysis, the removal of the old exoskeleton. The coordinated abdominal movements followed by ecdysis are often referred to in the art as the ecdysis motor program. Ecdysis is considered to be prevented when the insect cannot complete the task of removing the exoskeleton. The prevention of ecdysis may be manifested by failure of the insect to emerge from a cocoon, or by failure of a cuticle to completely detach from the insect. The new cuticle generally hardens before the insect can use any form of struggling movements to remove itself from the old cuticle. When this occurs in newly emerging adults, the wings become permanently disfigured and can no longer function for flight activity.

The insects that can be used in the methods described herein include insects from Class Uniramia (Insecta). Preferably, the insects include members from the Orders Protura, Collembola, Thysanura, Diplura, Ephemeroptera, Odonata, Plecoptera, Grylloblatodea, Orthoptera, Phasmida, Dennaptera, Embioptera, Dictyoptera, Isoptera, Zoraptera, Psocoptera, Mallophaga, Anoplura, Hemiptera, Homoptera, Thysanoptera, Neuroptera, Coleoptera, Strepsiptera, Mecoptera, Siphonaptera, Diptera, Lepidoptera, Trichoptera, or Hymenoptera, more preferably, Neuroptera, Coleoptera, Strepsiptera, Mecoptera, Siphonaptera, Diptera, Lepidoptera, Trichoptera, or Hymenoptera, even more preferably Coleoptera, and Lepidoptera. Members of the Order Coleoptera preferably include the Superfamily Tenebrionoidea, more preferably, the Family Tenebrionidae. A preferred member of the Order Coleoptera is Tenebrio molitor. Members of the Order Lepidoptera preferably include Superfamily Pyraloidea and Superfamily Bombycoidea, more preferably, Family Crambidae (formerly Pyralidae) and Family Sphingidae. Preferred members of the Order Lepidoptera are M. sexta and Plodia interpunctella (Hübner).

The present invention also provides methods for preventing ecdysis in an insect. Preferably, the insect is killed. The method includes exposing an insect to an effective amount of a compound that binds a polypeptide. In some aspects, the polypeptide to which the compound binds is a mouse IgG, and in other aspects, the polypeptide to which the compound binds is a mouse IgG-like polypeptide. Mouse IgG and mouse IgG-like polypeptides are described herein.

M. sexta is considered in the art to be one of the model systems for the study of ecdysis in insects (Chapman, The insects: structure and function, fourth ed., Cambridge University Press, pp. 363-412 (1998), and Mesce and Fahrbach, Front Neuroendocrinol., 23, 179-199 (2002)), and the data presented in Example 1 showing the prevention of ecdysis in M. sexta after exposure to an anti-mouse IgG antibody indicates that other insects will respond to compounds that bind mouse IgG and/or mIgG-like polypeptide in the same way, i.e., ecdysis will be prevented. Moreover, the preliminary results disclosed in Example 3 indicate that exposure of two other species of insects to an anti-mouse IgG antibody results in the prevention of ecdysis. The two other species, Indian meal moths (Plodia interpunctella (Hübner)) and Mealworms (Tenebrio molitor), are not closely related to M. sexta. Indian meal moths belong to the Order Lepidoptera, Superfamily Pyraloidea, Family Crambidae (formerly Pyralidae), subfamily Phycitinae. They are distantly related to M. sexta (Order Lepidoptera, Superfamily Bombycoidea, Family Sphingidae). The Pyraloidea are classified in the larger Obtectomeran group, whereas Bombycoidea is classified in the larger Macrolepidopteran group (Minet, J., Entomologica scandinavica. 22:69 95(1991)). Mealworms belong to the beetle Order Coleoptera, Superfamily Tenebrionoidea, Family Tenebrionidae. Coleoptera is a holometabolous insect, but is not closely related to the order Lepidoptera, to which M. sexta belongs. Currently accepted ordinal relationships place Lepidoptera (moths & butterflies) in a group with caddisflies (Trichoptera) and scorpionflies (Mecoptera).

Accordingly, the methods for preventing ecdysis in an insect, preferably killing the insect, can be used on insects from Class Uniramia (Insecta). The Orders within the Class Uniramia (Insecta) are disclosed herein. Preferably, the Orders are Neuroptera, Coleoptera, Strepsiptera, Mecoptera, Siphonaptera, Diptera, Lepidoptera, Trichoptera, or Hymenoptera, more preferably Coleoptera or Lepidoptera. Members of the Order Coleoptera preferably include the Superfamily Tenebrionoidea, more preferably, the Family Tenebrionidae. Members of the Order Lepidoptera preferably include Superfamily Pyraloidea and Superfamily Bombycoidea, more preferably, Family Crambidae (formerly Pyralidae) and Family Sphingidae. In this aspect of the invention, the compound is administered orally, by injection or by infusion, more preferably, orally. Methods for administering a compound by injection or by infusion are disclosed herein. When administered orally, the compound may be applied to plants on which the insect feeds. It is well known in the art that neural tissue specific insecticides can be administered orally to insects, cross the blood-brain barrier to interact with neurons in the central nervous system, and are active.

A compound that binds a mouse IgG and/or a mouse IgG-like polypeptide may be formulated in ready-to-use solutions, suspensions, wettable powders, pastes, soluble powders, dusts, granules, and the like. Such formulations may be used in the customary manner, for example by watering, spraying, dusting, atomizing, scattering, foaming, brushing on and the like. If appropriate, a formulation containing a compound can be injected into the soil, or applied to the seed of a plant. A formulation containing a compound can be present as a mixture with other known insecticides.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Prevention of Ecdysis in M. sexta

Materials:

M. sexta (late fifth instars, or ‘wanderers’ (i.e., larvae where the dorsal vessel has appeared); 1 milliliter (ml) Tuberculin syringes with 30 gauge ½ inch needle; antibodies: rat anti-mouse IgG conjugated to Cy-5 or Cy-3, (Jackson ImmunoResearch, West Grove, Pa.), goat anti-mouse IgG conjugated to Cy-5 or Cy-3 (lot number 18009, Jackson ImmunoResearch), donkey anti-mouse IgG conjugated Cy-5 or Cy-3, (lot number 19703, Jackson ImmunoResearch); solution for sham injections (antibody buffer, but no antibodies); desheathing saline (per liter of solution, 8.18 g of NaCl, 0.37 g of KCl, 0.58 g of CaCl₂-2H₂O, 5.04 g of Glucose, and 1.19 g of HEPES, pH 7.4); cup of deionized water or ice to anesthetize animals by submersion. Distilled water was added to the antibodies as suggested by the manufacturer. It was this solution that was injected into the larvae. The antibodies for controls: anti-rabbit IgG (Jackson ImmunoResearch); chrompure bovine serum albumin (BSA, Jackson ImmunoResearch); BSA conjugate of Cy-5 or Cy3. All antibodies were affinity purified. Cy-3 and Cy-5 refer to Indocarbocyanine and Indodicarbocyanine, respectively. The antibody buffer was 0.01 M sodium phosphate, 0.25M sodium chloride (pH 7.6). The concentrations of the antibodies were between about 1 mg/ml to about 1.8 mg/ml. Methods:

Larvae of M. sexta (Lepidoptera:Sphingidae) were reared on an artificial diet and maintained on a long-day photoperiod regimen (light:dark 17:7) at 27° C. and 50%-60% relative humidity. Developing pupae and pharate adults were maintained according to Amos and Mesce (Int. J. Insect Morphol. Embryol., 23, 27-37 (1994)), and were staged according to previously published criteria (Tolbert et al., J. Neurosci 3, 1158-1175 (1983); Amos and Mesce, Int. J. Insect Morphol. Embryol., 23, 27-37 (1994)) with development to the adult requiring ca. 18 days from the onset of pupation. Ages of animals are described in relation to specific events such as ecdysis, which is the shedding of the old cuticle. For example, a pupa on the day of ecdysis is referred to as a pupal day 0 (P-0) animal, and 1 day later as a (P-1) animal. Although developing adults are also given the pupal designator (e.g. P-16), pupal development is restricted to the first few days following pupal ecdysis, when the animal has the ability to enter diapause. Both male and female animals were used during the course of this study and the sex of each animal was noted.

The insect was submerged in D1 water for about 5 minutes until it stopped struggling and is relaxed when removed from the bath. The animals were removed from the water and laid flat on a table with dorsal side up. A needle was inserted into the dorsal vessel of the 9^{th} segment from head. The antibody was injected into the insect slowly over a time period of 15 seconds. The needle was removed, and the animal placed in a glass tube. The tube was labeled and placed into an incubator. The status of the insects was checked frequently during each day. Typically the amount injected into an M. sexia insect was between 25 microliters (μl) to 250 μl.

Results:

Six M. sexia larva were injected with 50 μl to 200 μl of antibody buffer containing 50% glycerol, 2 were injected with 50 μl desheathing saline, and 2 were injected with 200 μl desheathing saline. These 10 insects underwent normal ecdysis. Three larva were injected with 200 μl of donkey anti-mouse IgG, and all three displayed incomplete ecdysis. Another 3 larva were injected with 200 μl of donkey anti-mouse IgG, and 2 displayed incomplete ecdysis while the third underwent complete ecdysis. Three larva were injected with 100 μl (140 μg) of goat anti-mouse IgG, and 2 displayed incomplete ecdysis while the third underwent complete ecdysis. Three other larva were injected with 50 μl of goat anti-mouse IgG, and 2 displayed incomplete ecdysis while the third underwent complete ecdysis. It is noteworthy that all of these affected insects had a sclerotized cuticle. This suggests that the ‘tanning’ hormone, bursicon, was able to be released into the blood and was effective in activating cuticular tanning.

### Example 2

### Determination of the Amino Terminal Sequence of the 27 KiloDalton Protein From Cells of the CCAP 27/704 Cell Group

Materials

M. sexta fifth instar and older; peroxidase sheep anti-mouse IgG antiserum (Jackson ImmunoResearch); Trizma/Np40 solution (per liter of solution, 7.58 g of Trizma (Tris(hydroxylmethyl)aminomethane and Tris (Hydrochloride)) (Sigma, St. Louis, Mo.), 5.84 g of NaCl, and 1% of Nodinet P-40 (Sigma), pH 7.4); desheathing saline; hypomillinigs (per liter of solution: 0.37 g of KCl, 0.58 g of CaCl₂-2H₂O, 5.04 g of Glucose, and 1.9 g of HEPES, pH 7.4); and hypomillonigs plus protein inhibitors (to make 5 ml, 10 μl of 0.5 M EDTA, 20 μl of 0.057 M PMSF, 1 mg/ml Pepstatin, 1 mg/ml Aprotinin, and 1 mg/ml Leupeptin). Immunopure immoblized protein G was obtained from Pierce Biotechnology, Inc. (Rockford, Ill.), and buffers for use with the immoblized protein G were: 0.1 M Glycine (pH 2.4), 0.1 M sodium acetate (pH 5.0), 500 mM Tris buffer (pH 9), and 0.1 M citric acid. The immoblized protein G is set up in a 2 ml column as suggested by the manufacturer.

Methods:

The methods used to isolate a 27 kDa IgG-like protein from the larval M. sexta central nervous system is essentially as described by Klukas et al. (Microscopy Res. Tech., 35, 242-264 (1996)). Nerve cords of the M. sexta were dissected and transferred into 10 ml of desheathing saline on ice. The cords were pelleted in a microfuge for 10 minutes, and hypomillonigs containing protease inhibitors was added to the pellet until the final combined volume was 5 mls. The pellet was sonicated using a Branson Sonifier 250 (Danbury, Conn.) with an output cycle setting 3 and duty cycle 40 for approximately 5 minutes until the cords were disrupted. The disrupted cords were repelleted at 8000 rpm for 30 minutes in a Beckman model J2-21 (JA20 rotor) centrifuge at a temperature setting of 10° C. The first supernatant is the cytoplasm fraction, and was kept for testing.

Trizma/Np40 solution was added to the pellet for a final volume of 10 mls. The pellet was resuspend then sonicated. After pelleting at 8000 rpm for 30 minutes the supernatant was harvested. This second supernatant or Trizma/Np40 soluble fraction was the membrane fraction. The proteins binding to Protein G were isolated as suggested by the manufacturer. Briefly, the Trizma/Np40 soluble fraction was poured over an Immunopure immoblized protein G column that had been freshly washed with 8 mls of binding buffer (0.1 M sodium acetate, pH 5.0). Five milliliters of binding buffer were added to the Trizma/Np40 soluble fraction. This was placed on top of the column just as the binding buffer wash reached the top of the beads, but before the beads became dry in appearance. One milliliter fractions were collected. The first fractions eluted were the non-bound fraction. When Trizma/Np40 soluble fraction just reaches the top of the beads, 10 mls of binding buffer was added to the column to wash all the non-bound material from the column. The elution of material from the column was followed with a spectrophotometer at 260 nanometers (nm). Elution buffer (0.1 M Glycine, pH 2.4) was applied to release bound material. The column was washed with neutralizing buffer (500 mM Tris buffer, pH 9) to neutralize.

The protein concentration of each fraction collected during the isolation was determined by the BCA protein assay (Pierce Biotechnology Inc., Rockford, Ill.). Those fractions collected after addition of elution buffer contained the protein of interest.

The fractions containing eluted protein were resolved on 7.5% polyacrylamide gels, and the portion of the gel containing the 27 kDa protein was separated from the remainder of the gel. The amino terminal sequence of the 27 kDa protein present in the gel was determined by the Harvard Microchemistry Facility (Department of Molecular and Cellular Biology, Harvard University).

Results:

The amino terminal sequence of the 27 kDa protein was found to be XXA(Q/K)(Q/K)GVD(L/1)SR (SEQ ID NO:1), where each X is either (A/L) and (A/L), or (P/S) and (P/S). Further, the presence of two amino acids in parentheses and divided by a slash indicates that one of the two amino acids is present in that position. For instance, (A/L) indicates that either an alanine or a leucine is present in that position.

### Example 3

### Prevention of Ecdysis

The procedure of Example 1 was followed; however, glass needles were used to deliver the antibody solution, and the insects were anesthetized with carbon dioxide. The indian meal moths and mealworms used were at the final larval instar, and were injected on the dorsal side at the midline. The volume of solution to inject was initially determined by dividing the volume injected into the M. sexta by the weight of the M. sexta injected in Example 1, and determining a ratio of a certain number of microliters per unit weight. The body weight of the indian meal moths and mealworms to be injected here was determined, and the volume to inject was determined using the ratio described above. The antibodies were obtained from Jackson ImmunoResearch.

Plodia interpunctella (Hübner) (Indian meal moth) In an initial experiment, 5 larva were injected with 5011 of saline, and all these larva survived 24 hours but died by 48 hours. In a separate experiment, 13 larva were injected with 10 μl, 25 μl, or 50 μl antibody buffer alone. Seven survived 48 hours; 5 survived more than 48 hours but subsequently died, 1 survived 8 days, made a cocoon and developed into an adult but did not emerge. One larva that was handled but did not have an injection, survived and became a moth. Three were injected with between 10 and 20 μl rabbit-anti-mouse IgG conjugated to Cy5, and 1 of the 3 survived, became a pupa but did not make a cocoon and never emerged. Another 3 were injected with between 10-20 μl goat-anti-mouse Cy3; 1 of 3 survived and made a cocoon, became a pupa, and emerged. Three additional larva were injected with between 10-20 μl of goat-anti-Rabbit Cy3. One of these survived and did not make a cocoon, became a Pupa, and never emerged.

In another experiment, of 4 that were injected with 1-3 μl of antibody buffer containing 50% glycerol all died. Four other larva were injected with 1-3 μl of mouse IgG; 3 died, and 1 formed an adult moth. Four larva were injected with 10-20 μl goat-anti-mouse IgG (constant region), and 2 died, one failed to make a cocoon, and one went on to form an adult moth. Three larva were injected with goat anti-mouse IgG (lot number 46647). One died, 1 completed ecdysis, and 1 formed a cocoon and then displayed incomplete ecdysis.

Tenebrio molitor (Mealworms)

In the first experiment, 4 larva were injected with 5 IgG of mouse IgG, and 1 died while the other 3 became adults. Of 3 injected with 5 μl of goat anti-mouse (lot number 46647), all died.

In another experiment, 4 were injected with between 2 μl and 5 μl of mouse IgG. Ten days later, 2 had become pupa and later became adults. One had become a deformed beetle, and 1 had remained a larva and not changed. Three larva were injected with between 2 μl and 5 μl of donkey anti-mouse IgG (lot number 21605), and 1 became a beetle, 1 became a pupa but died with the cuticle attached to the posterior, and 1 displayed incomplete ecdysis.

In conclusion, preliminary results in M. sexta showed that anti-mIgG-injected insects (9 of 12) displayed an inability to undergo normal ecdysis behavior. It is noteworthy that all of these affected insects had sclerotized cuticle. This suggests that the ‘tanning’ hormone, bursicon, was able to be released into the blood and was effective in activating cuticular tanning. Preliminary results in two species of insects, not considered closely related to M. sexta, demonstrate that only anti-mIgG-injected insects displayed an inability to undergo normal ecdysis behavior, which was characterized by the insect being trapped in its cuticle. It is noteworthy that these affected insects had sclerotized cuticle, similar to the result observed in M sexta. None of the insects injected with other substances showed this outcome.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method for identifying a compound that prevents ecdysis comprising:
providing a compound that binds a mouse IgG;
exposing an insect to an effective amount of the compound; and
observing ecdysis in the insect, wherein failure of the insect to complete ecdysis indicates the compound prevents ecdysis.

2. The method of claim 1 wherein the compound binds an Fc domain of the mouse IgG antibody.

3. The method of claim 1 wherein the compound binds an F(ab′)2 domain of the mouse IgG antibody.

4. The method of claim 1 wherein the compound is a polypeptide.

5. The method of claim 4 wherein the compound is an antibody.

6. The method of claim 5 wherein the antibody is a polyclonal antibody or a monoclonal antibody.

7. The method of claim 1 wherein the insect is at a larval stage of development.

8. A method for identifying a compound that prevents ecdysis comprising:
providing a compound that binds a polypeptide obtainable from an insect, the polypeptide comprising an amino terminal sequence of X₁X₂A(Q/K)(Q/K)GVD(L/1)SR (SEQ ID NO:1), where X₁ is either alanine, leucine, proline, or serine and X₂ is either alanine, leucine, proline, or serine, and a molecular weight of between about 25 kDa and about 29 kDa;
exposing an insect to an effective amount of the compound; and
observing ecdysis in the insect, wherein failure of the insect to complete ecdysis indicates the compound prevents ecdysis.

9. The method of claim 8 wherein the compound is a polypeptide.

10. The method of claim 9 wherein the compound is an antibody.

11. The method of claim 10 wherein the antibody is a polyclonal antibody or a monoclonal antibody.

12. The method of claim 8 wherein the insect is at a larval stage of development.

13. A method for preventing ecdysis comprising:
exposing an insect to an effective amount of a compound that binds a mouse IgG antibody, wherein the insect does not complete ecdysis.

14. The method of claim 13 wherein the compound binds an Fc domain of the mouse IgG antibody.

15. The method of claim 13 wherein the compound binds an F(ab′)2 domain of the mouse IgG antibody.

16. The method of claim 13 wherein the compound is a polypeptide.

17. The method of claim 16 wherein the compound is an antibody.

18. The method of claim 17 wherein the antibody is a polyclonal antibody or a monoclonal antibody.

19. The method of claim 13 wherein the insect is at a larval stage of development.

20. A method for preventing ecdysis comprising:
exposing an insect to an effective amount of a compound that binds a polypeptide present in the insect, the polypeptide comprising an amino terminal sequence of X₁X₂A(Q/K)(Q/K)GVD(L/1)SR (SEQ ID NO:1), where X₁ is either alanine, leucine, proline, or serine and X₂ is either alanine, leucine, proline, or serine, and a molecular weight of between about 25 kDa and about 29 kDa, wherein the insect does not complete ecdysis.

21. The method of claim 20 wherein the compound is a polypeptide.

22. The method of claim 21 wherein the compound is an antibody.

23. The method of claim 20 wherein the antibody is a polyclonal antibody or a monoclonal antibody.

24. The method of claim 20 wherein the insect is at a larval stage of development.

25. A method for killing an insect comprising:
exposing an insect to an effective amount of a compound that binds a mouse IgG antibody, wherein the compound causes the insect to die after failing to complete ecdysis.

26. The method of claim 25 wherein the compound binds an Fc domain of the mouse IgG antibody.

27. The method of claim 25 wherein the compound binds an F(ab′)2 domain of the mouse IgG antibody.

28. The method of claim 25 wherein the compound is a polypeptide.

29. The method of claim 28 wherein the compound is an antibody.

30. The method of claim 29 wherein the antibody is a polyclonal antibody or a monoclonal antibody.

31. The method of claim 25 wherein the insect is at a larval stage of development.

32. A method for killing an insect comprising:
exposing an insect to an effective amount of a compound that binds a polypeptide present in the insect, the polypeptide comprising an amino terminal sequence of X₁X₂A(Q/K)(Q/K)GVD(L/1)SR (SEQ ID NO:1), where X₁ is either alanine, leucine, proline, or serine and X₂ is either alanine, leucine, proline, or serine, and a molecular weight of between about 25 kDa and about 29 kDa, wherein the compound causes the insect to die after failing to complete ecdysis.

32. The method of claim 31 wherein the compound is a polypeptide.

34. The method of claim 32 wherein the compound is an antibody.

35. The method of claim 33 wherein the antibody is a polyclonal antibody or a monoclonal antibody.

36. The method of claim 32 wherein the insect is at a larval stage of development.
